(19) United States
(12) Patent Application Publication
(10) Pub. No.: US 2009/0162334 A1
(43) Pub. Date: Jun. 25, 2009

(54) PRODUCTION AND USE OF REGULATORY T CELLS

(75) Inventors: Gang Feng, Oxfordshire (GB); Kathryn Jayne Wood, Oxfordshire (GB); Andrew Richard Bushell, Oxfordshire (GB)

Correspondence Address: BROOKS KUSHMAN P.C., 1000 TOWN CENTER, TWENTY-SECOND FLOOR, SOUTHFIELD, MI, 48075, US

(73) Assignee: ISIS INNOVATION LIMITED, Oxfordshire (GB)

(21) Appl. No.: 12/298,323

(22) PCT Filed: Apr. 24, 2007
(86) PCT No.: PCT/GB07/50210

§ 371 Date: Dec. 3, 2008

(30) Foreign Application Priority Data
Apr. 24, 2006 (GB) 0608054.3

(51) Int. Cl. A61K 35/12; C12N 5/06; C12N 5/08

(52) U.S. Cl. 424/93.71; 435/375; 435/372.3

(57) ABSTRACT

An ex vivo method for generating a population of Treg capable of suppressing rejection of an organ or tissue transplant from a donor animal, comprises culturing CD4⁺ T cells from a recipient animal in the presence of IFN-γ plus either donor specific or third-party antigen presenting cells, and harvesting a population of Treg capable of suppressing rejection in the recipient animal. The Treg can be administered, for example intravenously to the recipient, preferably immediately prior to the transplant to suppress transplant rejection. A similar strategy applicable to generating a population of Treg capable of suppressing an autoimmune condition in an animal wherein the animal mounts an immune reaction against an autoantigen, comprises culturing CD4⁺ T cells from the animal in the presence of cells presenting the autoantigen and IFN-γ and harvesting a population of autoantigen reactive Treg.

### FIELD OF THE INVENTION

This invention relates to the generation and/or expansion of populations of regulatory T cells and their use, for example in a cellular therapy for preventing the rejection of tissue and organ transplants.

### BACKGROUND OF THE INVENTION

Transplantation is the treatment of choice for end stage kidney, heart, liver and pancreas organ failure and despite considerable advances in the management of transplant rejection in recent years the vast majority of transplants are eventually rejected. In addition, the current immunosuppressive regimens which depend on continual drug therapy predispose transplant patients to increased susceptibility to infections and cancer because even the most sophisticated drugs are unable to inhibit just those responses directed toward the transplant. As a result opportunistic infection remains one of the main causes of mortality in heart transplant patients and predictive calculations have shown that 30 years of continual immunosuppression carries a 100% risk of some types of cancer. In many animal transplant models it is possible to achieve indefinite transplant survival by transient manipulation of the recipient immune system and in many of these situations regulatory cells develop with time such that they prevent rejection even after cessation of the initial therapy. Waldmann and Cobbold¹ discuss the developments over recent years that have led to the possibility of providing short-term therapy for long-term tolerance of organ grafts.

CD4⁺ T-helper lymphocytes are cells of the immune system and in normal situations play an essential role in immune responses that protect us from pathogenic organisms such as bacteria and viruses. In the context of transplantation however, these same cells are largely responsible for the rejection of organ transplants. It is widely known that rejection responses can be attenuated by administration of immunosuppressive agents, including anti-CD4 antibody which targets CD4⁺ T cells, but in recent years it has been shown that such antibody therapy can lead to the generation of sub-populations of T cells with the capacity to control or regulate destructive rejection responses. It is believed that regulatory cells arise in such situations because the presence of the anti-CD4 antibody prevents full T cell activation and the cells default to a regulatory or suppressive phenotype.

The existence of lymphocytes with suppressive capacity was first described over thirty years ago², but in recent years there has been renewed interest in the identification and characterisation of such regulatory T cells (T-reg). Several cell surface markers have been identified that enrich for regulatory activity, one of which is CD25, the α subunit of the IL-2 receptor. CD25⁺CD4⁺ T-reg with the capacity to regulate responses in vitro have been identified in both mice³⁻⁷ and humans⁸⁻¹³. T-reg can suppress the proliferation and/or effector activity of both CD4^{+3,5} and CD8^{+4,6,14,15} T cells, can prevent the development of autoimmune disease¹⁶⁻¹⁸, and have been shown to play a role in both tumour immunity^{19,20} and transplantation^{14,21-25}. In vivo, but not in vitro, regulatory activity can be dependent on IL-10²⁶, TGF-β²⁷, and CTLA-4^{27,28}. In vitro studies with mouse cells have demonstrated that, although these regulatory populations require activation via their T cell receptors in order to regulate, once activated they can inhibit responses in an antigen non-specific manner, the process of ‘bystander regulation’^{3,5,7}.

The presence of T-reg with the capacity to suppress allograft rejection has been demonstrated in rodents with long term surviving cardiac^{14,21,22} and pancreatic islet^{23,24} allografts. It has previously been shown that pre-treatment of mice with donor-specific blood transfusion under the cover of anti-CD4 antibody allows the acceptance of fully allogeneic cardiac grafts²⁹. Using an adoptive transfer system it has been shown that pre-treatment of CBA (H2^{k}) mice with transfusion of blood from B10 (H2^{b}) mice under the cover of the anti-CD4 antibody YTS177 generates CD25⁺CD4⁺ cells that prevent rejection of donor-type skin allografts mediated by CD45RB^{high}CD4⁺ effector cells. Significantly, equal numbers of CD25⁻CD4⁺ cells from pre-treated animals or of CD25⁺CD4⁺ cells from naïve mice or from mice pre-treated with antibody or transfusion alone were unable to regulate in this manner, demonstrating that these T-reg arise entirely as a consequence of the full pre-treatment protocol²⁵. In common with naturally occurring CD25⁺CD4⁺ T-reg, regulation by these alloantigen-induced cells is dependent on IL-10 and CTLA-4²⁵.

In recent years it has become clear that populations of Treg play an essential role in controlling normal immune responses, for example in preventing autoimmune disease. It has been shown in rodent models that it is possible to generate/expand populations of Treg in vivo that can prevent transplant rejection providing a proof-of-concept for the potential of such cells in transplantation. However, the generation of these cells in vivo depends on manipulation of the recipient's immune system which may result in side effects similar to those associated with conventional immunosuppression. An alternative approach would be to generate such cells ex vivo and then administer them to the recipient as a cellular therapeutic. Several methods have been described for generating/expanding Treg ex vivo but most require that the responding populations are further selected by sophisticated cell sorting techniques (usually by fluorescence activated cell sorting, FACS).

WO 2004/112832 describes an ex vivo method for generating a Treg population which comprises culturing T cells with an antibody directed at a cell surface antigen selected from CD4, CD8, CD154, LFA-1, CD80, CD86 and ICAM-1, in the presence of cells that present alloantigen. The T cells can be from the recipient of an organ or tissue transplant and the alloantigen can be from donor.

Several other reports have appeared in the literature of attempts to generate Treg ex vivo³⁰⁻³⁵. In addition, it has been shown by Bocek et al³⁶ that interferon-γ (IFN-γ) can enhance IL-4 production by CD4⁺ T cells but the report contains no in vivo functional data and, more importantly, the aim was to stimulate IL-4 production.

Hong et al³⁷ used a model involving copolymer-1 (COP-1) which is a random polymer of four amino acids found particularly in myelin basic protein (MBP) in the generation of Treg. It is know that MBP is one of the targets of auto-reactive T cells thought to be closely involved in the neuronal degeneration seen in multiple sclerosis patients and the interest in COP-1 was to use this peptide mix to generate Tregs that might influence the progression of the disease. Hong et al showed that human CD4⁺ T cells stimulated ex vivo by COP-1 in the presence of autologous antigen presenting cells without other additions up-regulate the expression of the transcription factor Foxp3.

Foxp3 expression is known to be highly associated with the generation/function of Treg and so the authors interpreted their observations to mean that COP-1 stimulation drives Treg generation. They further showed that COP-1 stimulates the production of IFN-γ, TGF-β and TNF-α and more importantly, that addition of recombinant IFN-γ to total peripheral blood mononuclear cells (PBMC) results in Foxp3 induction. However, only phenotypic data linking IFN-γ with Foxp3 expression was disclosed. The authors administered COP-1 to normal mice and to those deficient for IFN-γ (IFN-KO mice), harvested CD4⁺ CD25⁺ T cells to determine whether these could inhibit the responses of normal T cells polyclonally stimulated with anti-CD3 plus anti-CD28 antibodies. Cells taken from normal mice inhibited the proliferation whereas cells from IFN-KO mice did not and the implication was that IFN-γ was essential for the generation of Treg in vivo. The aim of the Hong et al paper was to generate Tregs using COP-1, a peptide mixture. The authors infer that their cells are regulatory cells based just on phenotypic data (up regulation of Foxp3 expression) and although they show regulation in vitro, T cell activation in general can result in Foxp3 expression and many different strategies can lead to T cells that regulate in vitro without concomitant regulation in vivo. Furthermore, Hong et al used syngeneic (self) antigen presenting cells and they did not disclose functional in vivo results.

An object of the present invention is to provide a method of generating and/or expanding donor-reactive Treg populations without the need for sorting thereby providing a significant improvement on many current strategies.

A further object of the invention is to provide a method of generating and/or expanding autoantigen reactive Treg capable of suppressing an autoimmune condition.

### SUMMARY OF THE INVENTION

According to one aspect, the present invention provides an ex vivo method for generating a population of Treg capable of suppressing rejection of an organ or tissue transplant from a donor animal in a recipient animal, which method comprises culturing CD4⁺ T cells from the recipient animal in the presence of IFN-γ plus either donor specific or third-party antigen presenting cells, and harvesting a population of Treg capable of suppressing rejection in the recipient animal.

According to another aspect, the present invention relates to a method of suppressing rejection of an organ or tissue transplant from a donor animal in a recipient animal comprising the following steps:

(i) obtaining a sample of CD4⁺ T-cells from the recipient animal;
(ii) culturing the said CD4⁺ T cells in the presence of IFN-γ plus either donor specific or third party antigen presenting cells;
(iii) harvesting a population of Treg from said culture capable of suppressing rejection in the recipient animal; and
(iv) administering said Treg to the recipient animal.

According to a further aspect, the present invention provides an ex vivo method for generating a population of Treg capable of suppressing an autoimmune condition in an animal wherein the animal mounts an immune reaction against an autoantigen, which method comprises culturing CD4⁺ T cells from the animal in the presence of cells presenting the autoantigen and IFN-γ and harvesting a population of autoantigen reactive Treg.

According to a still further aspect, the present invention relates to a method of suppressing an autoimmune condition in an animal wherein the animal mounts an immune reaction against an autoantigen comprising the following steps:

(i) obtaining a sample of CD4⁺ T-cells from the animal;
(ii) culturing the said CD4⁺ T cells in the presence of cells presenting the autoantigen and IFN-γ;
(iii) harvesting a population of autoantigen reactive Treg from said culture; and
(iv) administering said Treg to the animal.

### BRIEF DESCRIPTION OF THE DRAWINGS

FIGS. 1A-D illustrate the use of IFN-γ conditioned cells to prevent skin allograft rejection in a mouse model.

FIGS. 2A-C illustrate the use of IFN-γ conditioned cells to prevent islets allograft rejection in a mouse model.

FIGS. 3A-C illustrate titration of IFN-γ in the conditioning protocol for various mouse strain combinations.

FIG. 4 illustrates that Foxp3 up-regulation driven by IFN-γ conditioning is abolished by iNOS inhibitor.

### DETAILED DESCRIPTION OF THE INVENTION

According to one aspect of the invention, Treg capable of suppressing rejection of an organ or tissue transplant from a donor in a recipient are generated and/or expanded ex vivo by culturing CD4⁺ T cells from the transplant recipient with IFN-γ plus either donor specific or third-party antigen presenting cells (APC). The population of Treg that can be derived from this culture is introduced into the transplant recipient (patient) for use in prevention of transplant rejection.

According to one particularly preferred embodiment of this aspect of the invention, the method is applied to transplantation involving a human donor and a human recipient.

The APC are preferably cells presenting donor specific antigen and are more preferably APC derived directly from the donor animal. However, in some cases it may be possible to achieve an equivalent effect using APC not directly derived from the donor animal for example using cells pulsed with antigen from the donor animal. Alternatively, as described in more detail below, it may be possible to use third-party APC, i.e. APC from genetically unrelated donors, to drive the generation of Treg that regulate in the donor by cross-reactivity or by non-specific “bystander regulation”.

The majority of regulatory T cells described in the literature thus far are CD4+ which in the context of allo-recognition implies that they recognize allogeneic MHC Class II via the direct pathway and/or allogeneic peptides presented by self Class II via the indirect pathway. Since direct pathway presentation probably dominates the acute immune response after transplantation it is theoretically desirable to generate regulatory T cells against intact allogeneic Class II molecules. Many different cell types express MHC Class II molecules including dendritic cells (DC), macrophages and B cells. However, the best type of cells for this type of presentation are DC which are thus preferred for use according to the invention.

Bone marrow is a rich source of DC and so where available bone marrow dendritic cells (BM-DC) are particularly preferred for use according to the invention. Thus in the case of experiments using mouse models, successful use has been made of DC precursors isolated from the bone marrow of donor strain mice.

It may also be possible to use the spleen as a source of APC although the proportion of DC in total spleen cells is relatively low so that the yield of Treg would also be low. This may be relevant in the case of living donor transplantation where obtaining BM-DC may be impracticable and in this case DC or other APC populations can be isolated from peripheral blood and used to stimulate CD4⁺ T-cells from the recipient. Since the identity of a living donor will be known well in advance of transplantation, this procedure can be undertaken ahead of transplantation so that Treg will be available for administration at the most advantageous time relative to the time of transplant.

Use of APC derived directly from the donor may be problematical in the case of cadaveric transplantation since APC, whether from bone marrow or peripheral blood, can generally only be harvested from the donor at the same time as organ harvest. In this case, whilst Treg could be generated ex vivo by the method according to the invention, they would only be available for use after transplant. Accordingly, in the case of cadaveric transplantation, APC from unrelated cell donors can be used to drive Treg generation. Thus, in the case of recipient “A”, it would be possible to simulate the generation of Treg from “A” by use of APC from say individuals “B”+“C”+“D” to regulate rejection of a graft from donor “Z”. It is anticipated that Treg generated in this situation regulate either by cross-reactivity (where some antigens presented by “B” or “C” or “D” are sufficiently similar to those on donor “Z”) or more probably by “bystander regulation” where Treg regulate in an antigen non-specific manner.

The normal role of DC is to present antigen to T cells in such a way that the T cells become activated so that for use according to the invention DC may need to be manipulated so that they present antigen but do not activate the T cells. Conditioning BM-DC with GM-CSF and TGF-β seems particularly effective in achieving this result and is thus preferred for use according to the invention. Without wishing to be bound by any particular theory, it is believed that GM-CSF enhances DC expansion and TGF-β may be capable of modifying DC so that they present antigen but do not activate the responding T cells. Conditioning with GM-CSF and TGF-β is not essential when using APC from the spleen.

According to a preferred embodiment, total CD4⁺ T cells from the recipient are exposed in tissue culture to GM-CSF and TGF-β conditioned donor-type bone marrow dendritic cells (BM-DC) in the presence of IFN-γ. After a sufficient period, generally several days, for example 5 to 10 days, preferably about 7 days, the cells are re-stimulated under identical conditions and harvested after a further sufficient period, again generally several days, for example 5 to 10 days, preferably about 7 days. The majority of input cells die (typically 70-90%) because they are not stimulated by the donor cells leaving a population enriched for donor-reactive T cells. In addition, the presence of IFN-γ drives the selection of cells no longer capable of producing the transplant-destructive cytokine interleukin-4 (IL-4). Thus, the resultant T cell population is selected for donor-reactive cells but depleted of those capable of tissue damage. These cells (referred to as IFN-γ conditioned T cells) thus respond to a transplant without causing damage and have the potential to inhibit destructive responses mediated by other T cell populations.

Regulatory T cells generated ex vivo can be administered to the recipient either before or after transplantation, preferably as a cell suspension in a suitable medium such as physiological saline. Intravenous administration, such as by intravenous infusion, is preferred although other modes of administration may be possible such as intraperitoneal administration or, in the case of certain types of transplant such as islet transplantation or skin transplantation, local administration at the graft site.

Administration of Treg a short time prior to transplantation, for example a day before, is preferred which makes the present invention particularly suitable for use in the case of live donation of the organ or tissue transplant. Administration after initiation of the rejection process has commenced is unlikely to be effective but a protocol could be envisaged in which Treg are administered at the optimal time point, i.e. shortly before transplantation, and Treg are re-administered subsequently if clinical indicators suggest a decline in graft function. In this case it would be appropriate for Treg to be continuously stimulated/expanded by the method according to the invention for an appropriate period after transplantation as a contingency against delayed rejection.

The appropriate dose of Treg will depend on the type of transplant and in the case of man as the recipient will be at the discretion of the attendant physician. In animal models a dose of about 2×10⁵ Treg has been used to control 1×10⁵ effector cells. The precise numbers of ex vivo generated Treg required to influence transplant rejection in man will be determined by carefully designed clinical trials but extrapolation from the mouse data suggests that doses in the range of 10⁹ to 10¹² Treg may be considered appropriate.

The recipient may be treated with additional immunosuppression or adjunctive therapy to attenuate any immediate rejection response that occurs. The additional immunosuppression or adjunctive therapy may comprise administration of a sub-therapeutic dose of an immunosuppressive agent, preferably an agent used in a manner (time/dose) that does not block the function of the regulatory T cells, in the immediate post-operative period. Suitable immunosuppressive agents or adjunctive therapies include treatment with an anti-CD8 antibody or with rapamycin. The intention is that the combination of the ex vivo generated Treg with a sub-therapeutic dose of an immunosuppressive agent would lead to the prolonged survival of fully allogeneic allografts, for example cardiac allografts, in fully immunocompetent recipients. A sub-therapeutic dose can be identified by reference to clinical studies identifying suitable therapeutic doses.

The use of ex vivo generated Treg according to the present invention may also be combined with preconditioning of the recipient (patient) to remove memory T cells which may be much more difficult to control than naïve T cells. There is good evidence to suggest that such cells can be a barrier to the induction of operational tolerance so that pre-elimination may be necessary. Several antibodies are currently available for use in transplantation should this type of conditioning be necessary and examples include anti-lymphocyte serum/anti-lymphocyte globulin, anti-CD3 antibodies, and anti-CD52 antibodies (such as CAMPATH-1H).

Donor reactive T-reg acquire the capacity to control the activity of graft destructive T cells so that transplant rejection can be suppressed or prevented provided that transplantation takes place whilst the T-reg are activated. Once transplantation has taken place, the protection provided by the T-reg would be maintained due to prolonged Treg activation provided by antigen presenting cells from the graft itself. Based on published data obtained in other mouse transplant models it is anticipated that regulation mediated by ex vivo generated Treg could lead to the additional generation in vivo of Treg which would contribute to and maintain operational tolerance⁴¹.

The phenomenon of regulation in the specific setting of transplantation is of interest because active, self-sustaining regulation of rejection responses can provide a route to drug-independent long-term graft survival. All transplants carried out clinically evoke powerful rejection responses mediated by cells of the immune system which if left unchecked result in destruction of the transplanted organ or tissue within a few days. Current clinical protocols to prevent rejection rely on powerful immunosuppressive drugs that must be administered indefinitely to provide continued graft survival. These drugs suppress the immune system in a non-selective manner leading to a variety of immunological and non-immunological complications including an increased risk of opportunistic infection, malignancy, vascular disease, hyperlipidemia and hypertension. By targeting specifically just those immune responses directed against the transplant, long-term graft survival could be achieved without the damaging side effects of current therapies.

A similar strategy to that described above with respect to transplantation can also be applied to autoimmune conditions. Thus, according to a further aspect of the invention, Treg capable of suppressing an autoimmune condition in an animal wherein the animal mounts an immune reaction against an autoantigen are generated and/or expanded ex vivo by culturing CD4⁺ T cells from the animal in the presence of cells presenting the autoantigen and IFN-γ. The population of Treg that can be derived from this culture can be introduced into the animal (patient) for use in prevention or alleviation of the autoimmune condition.

According to one particularly preferred embodiment of this aspect of the invention, the animal suffering from an autoimmune condition is man.

Generally Treg for the suppression of an autoimmune condition are produced in an analogous manner to Treg for the suppression of transplant rejection.

Examples of autoimmune conditions include rheumatoid arthritis, multiple sclerosis insulin-dependent diabetes melitus and inflammatory bowel disease. CD4⁺ T cells play a central role in autoimmunity and have the capacity to be both protective and pathogenic. Accumulating evidence suggests that autoimmunity probably results when normal regulatory functions of protective CD4⁺ T cells break down. Autoimmune diseases can be treated to a certain extent by manipulation of CD4⁺ T cells. However, the effects may be only transient due to T cell turn-over and re-acquisition of T cell function. If such recovering T cells re-encounter auto-antigens that initiated the initial disease during on-going inflammation of the target tissue (for example the synovial joint in rheumatoid arthritis, pancreatic β-cells in insulin-dependent diabetes), the T cells will become activated and autoimmune destruction will re-occur. It may be possible to re-establish a balance between pathogenic and protective T cells by transient therapy involving the intravenous administration of autoantigen reactive Treg.

The generation and/or expansion of a population of autoantigen reactive Treg involves use of cells presenting the autoantigen. These can be obtained as self-APC plus exogenous autoantigen or APC isolated from the site of autoimmune attack which can be assumed to be presenting autoantigen. In the case of autoimmune conditions where candidate autoantigens can be identified, self-APC can be pulsed (loaded) with the autoantigen. Examples of autoantigens which have been identified as associated with particular autoimmune conditions include myelin basic protein in multiple sclerosis and GAD69 in Type 1 diabetes and peptide fragments of these antigens, for example synthesised using recombinant DNA technology, can be used as the autoantigen. Where candidate autoantigens have not been identified, APC can be isolated from a site of autoimmune attack. Examples include APC from synovial fluid in the case of rheumatoid arthritis, APC from the pancreas or from draining lymphoid tissue in the case of Type 1 diabetes and APC from the gut and/or Peyers patches in the case of inflammatory bowel disease.

Preconditioning of the animal (patient) prior to treatment with autoantigen reactive Treg may be necessary to deplete autoantigen reactive T cells. For example, a promising approach for the control of Type 1 diabetes is to treat the patient with antibodies that target all T cells so that following this targeting the “immunological rheostat” is reset in a manner such that self-tolerance rather than self-reactivity prevails³⁸. Antibodies such as those referred to above for preconditioning in the context of transplantation, and in particular humanised anti-CD3 and humanised anti-CD52 antibodies, may also be useful in the context of preconditioning patients with autoimmune conditions.

### EXAMPLES

The invention is based on and illustrated by the following experimental work.

### Example 1

### In Vitro Polarization and Conditioning Protocol

Purified naive recipient CD4⁺ T cells are co-cultured with donor BM-DC for 7 days in standard tissue culture medium (RPMI 1640 containing 10% foetal calf serum, glutamine and antibiotics—‘complete medium’) in the presence of 5 ng/ml IFN-γ. All incubations are carried out at 37° C. in an humidified CO₂ gassed incubator. At day +7 the cells are harvested, washed in RPMI 1640 medium and re-stimulated with fresh BM-DC in the presence of IFN-γ as described above. At day +12, the cells are harvested, washed in RPMI 1640 medium, re-suspended in phosphate buffered saline and used in vivo as described.

Bone marrow (BM) derived DCs are generated from B10 donors using a modification of published methods^{39,40}. Briefly, bone marrow cells are flushed from isolated mouse femurs using RPMI 1640. Red blood cells are lysed by hypotonic shock; and B cells, T cells, and MHC class II positive cells are depleted using cell-specific antibodies followed by negative selection using anti-rat magnetic beads. Enriched DC precursor cells are placed in 24-well plates in 1 ml of complete medium supplement with 2 ng/ml each of recombinant mouse granulocyte/monocyte colony stimulating factor (rmGM-CSF) and recombinant human transforming growth factor-β (rhTGF-β). 75% of the medium is replaced with same every 48 hours, and at day 6, BM-DC are harvested, washed and irradiated (3000 rads from a sealed Cs source) prior to use.

Donor-APC can also be isolated from lymphoid tissue (spleen, lymph nodes) or from peripheral blood using established methods. When these APC are used to drive Treg generation they are incubated with purified recipient CD4+ T cells in the presence of IFN-γ as described above but with the further addition of 40 ng/ml recombinant interleukin-10 (rIL-10). It should be noted however that the yield of Treg obtained using these ‘peripheral’ APC is considerably reduced compared to that obtained using GM-CSF/TGF-β derived BM-DC.

### Example 2

### IFN-γ Conditioned Cells Prevent Skin Allograft Rejection

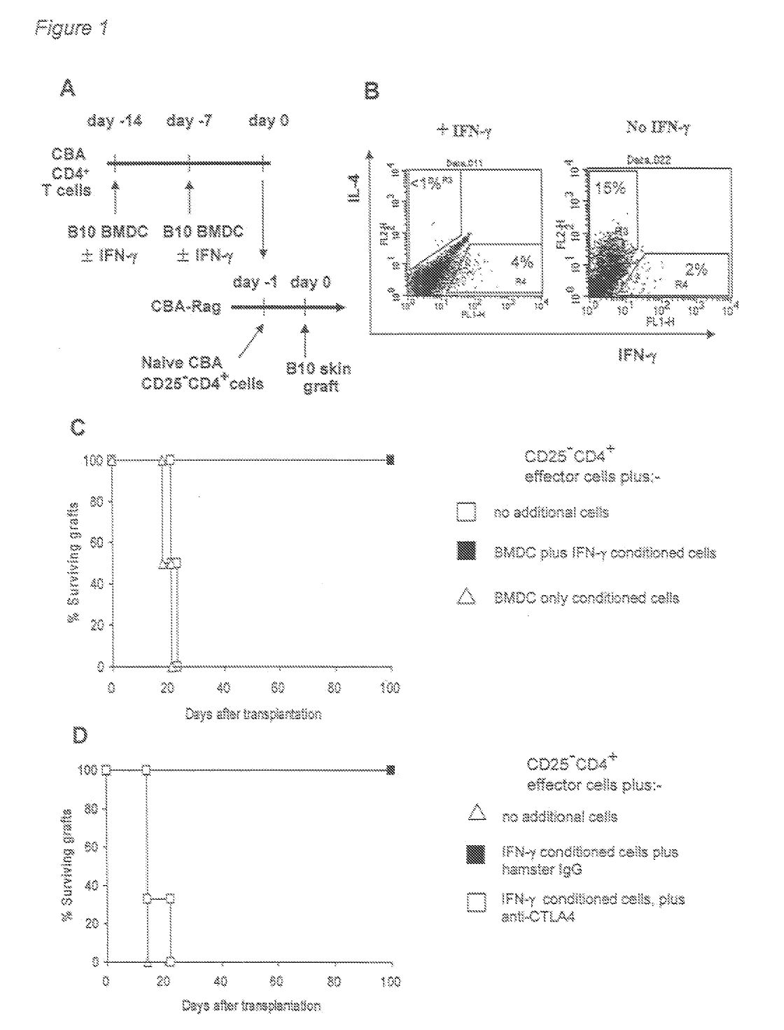

FIG. 1A shows the conditioning and adoptive transfer protocol. All CBA-Rag^{-/-} mice were reconstituted with 10⁵ CD25⁻CD4⁺ cells from naïve CBA mice, with or without conditioned cells. The reconstituted mice then received a B10 skin graft the following day.

FIG. 1B shows that IL-4 producing cells are virtually undetectable by intracellular staining.

FIG. 1C shows the effect of conditioned cells on CD25⁻CD4⁺-mediated rejection of B10 skin grafts. Mice reconstituted with 10⁵ CD25⁻CD4⁺ cells alone acutely rejected B10 skin grafts (□; MST=22 days, n=2). Cotransfer of 4×10⁵ IFN-γ conditioned cells prevents rejection of B 10 skin grafts (▪; MST>100 days, n=4), whereas cotransfer of 4×10⁵ cells driven by BMDC in the absence of IFN-γ only conditioned cells did not prevent rejection (Δ; MST=19.5 days, n=4).

FIG. 1D shows the same protocol as depicted in FIG. 1A, except that 0.8 mg of anti-CTLA4 antibody or control antibody was given at the time of cell transfer and weekly thereafter for 4 weeks or until rejection was observed. Mice reconstituted with 10⁵ CD25⁻CD4⁺ cells alone acutely rejected B10 skin grafts (Δ; MST=14 days, n=4). Cotransfer of 2×10⁵ IFN-γ conditioned cells and treated with anti-CTLA4 mAb acutely rejected B10 skin grafts (□; MST=16.7 days, n=3), whereas administration of control antibody had no effect on the ability of cotransfered IFN-γ conditioned cells to prevent rejection (▪; MST>100 days, n=4).

### Example 3

### IFN-γ Conditioned Cells Prevent Islets Allograft Rejection

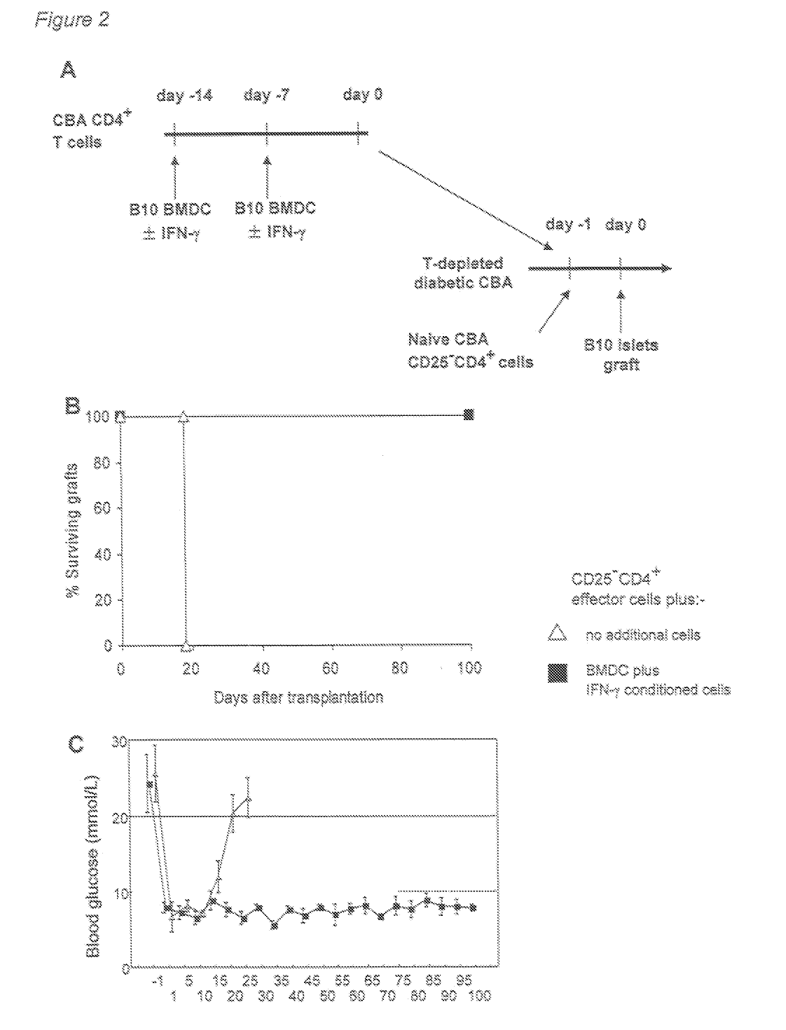

FIG. 2A shows the conditioning and adoptive transfer protocol. T cell depleted mice were rendered diabetic with streptozotocin at day −10 and reconstituted with 10⁵ CD25⁻ CD4⁺ cells from naive CBA mice, with or without 4×10⁵ IFN-γ conditioned cells. The reconstituted mice then received 400 B10 islets graft the following day.

FIG. 2B shows the effect of conditioned cells on CD25⁻CD4⁺-mediated rejection of B10 islets grafts. Mice reconstituted with 10⁵ CD25⁻CD4⁺ cells alone acutely rejected B10 islets grafts (Δ; MST=18 days, n=2). Cotransfer of 4×10⁵ IFN-γ conditioned cells prevent rejection of B10 islets grafts (▪; MST>100 days, n=3).

As shown in FIG. 2C, graft function was evaluated with daily glucose measurements and graft rejection was defined as blood glucose >14.5 mmol/L.

### CONCLUSIONS

FIG. 1 shows skin graft survival mediated by Treg generated according to the invention and FIG. 2 shows that Treg generated according to the invention also prevent rejection of a life-sustaining islet transplant under physiological load. Both experiments used an adaptive transfer model where rejection is mediated by a relatively small number of cells. With transfer of effector cells only, skin grafts were rejected at about day 15 and islets at about day 20. In the case of the skin grafts, the grafts simply become necrotic and form a scab whereas in the case of the islets the mice become hyperglycemic (diabetic) and will die. Co-transfer of 2×10⁵ ex vivo generated Treg prevents this rejection in both situations and in the case of the islet model results in stable normal blood glucose for >100 days (the point at which the experiment was terminated).

### Example 4

### Titration of IFN-γ in Various Strain Combinations

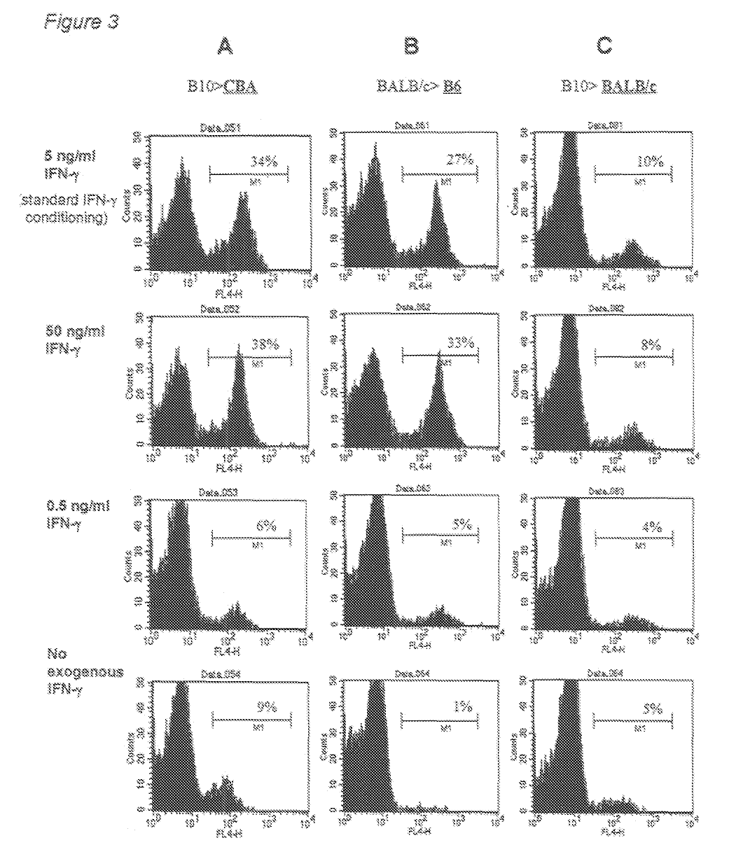

FIG. 3 shows that the IFN-γ conditioning protocol increases the proportion of cells that express Foxp3 in a dose-dependent manner in two of three strain combinations examined. Foxp3 is a transcription factor whose expression is highly (though not exclusively) associated with regulatory T cells and is considered at present to be the best ‘identifier’ of regulatory T cells. Since in vivo data have shown that the IFN-γ conditioning protocol generates/selects T cells that regulate allograft rejection, it is predicted that the acquisition of such function would be associated with an increase in Foxp3 expression. This prediction is confirmed by the data in FIG. 3. The experiments shown in FIG. 3 were as follows:

(A) CBA CD4⁺ T cells were conditioned with GM-CSF/TGF-β differentiated C57BL/10 (B10) BM DCs and IFN-γ (0.5-50 ng/ml).
(B) C57BL/6 (B6) CD4⁺ T cells were conditioned with GM-CSF/TGF-β differentiated BALB/c BM DCs and IFN-γ (0.5-50 ng/ml).
(C) BALB/c CD4⁺ T cells were conditioned with GM-CSF/TGF-β differentiated C57BL/10 (B10) BM DCs and IFN-γ (0.5-50 ng/ml).

After two rounds of stimulation, harvested conditioned cells were stained for Foxp3 expression.

The data take the form of FACS histograms where Foxp3 expression is plotted on the x-axis and cell number on the y-axis. The bigger the size of the peak in the region denoted M1, the greater is the proportion of cells expressing Foxp3. Panel A shows the proportion of Foxp3 +ve cells in the absence of exogenous IFN-7 and at 0.5, 5 and 50 nm/ml. The basal proportion (no exogenous IFN-γ) is 9% and this is unaltered in the presence of 0.5 ng/ml IFN-γ. However, in the presence of 5 ng/ml IFN-γ—the concentration used in the protocol described herein—there is an almost 4-fold increase in the % of Foxp3 positive cells. Increasing the concentration 10-fold has little additive effect.

As shown in FIG. 3, panel A shows data for cells of the CBA mouse strain responding to antigen presenting (stimulator) cells of the B.10 strain. Panel B shows that when responder cells of the B6 strain are used and stimulated with cells of the BALB/c strain there is a similar increase in Foxp3 expression showing that this phenomenon is not restricted to a single stimulator-responder combination and giving confidence that this approach for generating regulatory T cells may also be applicable to human cells. The same increase was not seen when using BALB/c responders and B.10 stimulators, suggesting that further optimisation of the system for this combination may be required.

### Example 5

### Foxp3 Up-Regulation Driven by IFN-γ Conditioning is Abolished by iNOS Inhibitor

There is good reason to believe that the generation of regulatory T cells by IFN-γ might involve nitric oxide (NO). In order to test this it is possible to use a compound that inhibits the enzyme that produces NO (inducible nitric oxide synthase, iNOS).

Purified naive CBA CD4⁺ T cells were cocultured with GM-CSF/TGF-β differentiated B10 DCs in the absence or presence of different concentrations of L-NMMA (0.1-1.0 mM). IFN-γ (5 ng/ml) was used in all cultures. Cells were harvested on day 14 and intracellular Foxp3 expression analyzed. Populations were gated on CD4⁺ T cells. (L-NMMA=N-methyl-L-arginine, iNOS inhibitor.)

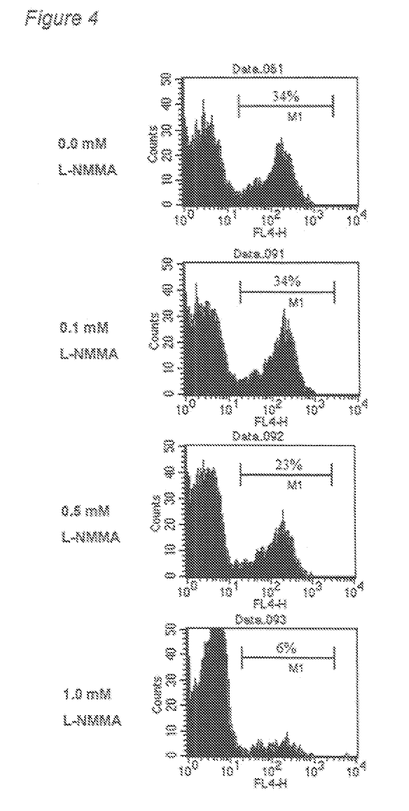

FIG. 4 shows that when the inhibitor L-NMMA is added to our IFN-γ conditioning cultures at concentrations ranging from 0.1 to 1 mM, the proportion of Foxp3 positive cells recovered is reduced in a dose dependent manner with 1 mM inhibitor reducing the Foxp3 positive proportion to essentially basal levels. These data provide additional mechanistic insights into the generation of regulatory T cells in this system.

### REFERENCES

1. Waldmann, H. & Cobbold, S. Approaching tolerance in transplantation. Int. Arch. Allergy Immunol. 136, 11-22 (2001)
2. Gershon, R. K. & Kondo, K. Infectious immunological tolerance. Immunology 21, 903-914 (1971).
3. Takahashi, T. et al. Immunologic self-tolerance maintained by CD25+CD4+ naturally anergic and suppressive T cells: induction of autoimmune disease by breaking their anergic/suppressive state. Int. Immunol. 10, 1969-1980 (1998).
4. Gao, Q., Rouse, T. M., Kazmerzak. K. & Field, E. H. CD4+CD25+ cells regulate CD8 cell anergy in neonatal tolerant mice. Transplantation 68, 1891-1897 (1999).
5. Thornton. A. M. & Shevach, E. M. Suppressor effector function of CD4+CD25+ immunoregulatory T cells is antigen nonspecific. J. Immunol. 164, 183-190 (2000).
6. Piccirillo, C. A. & Shevach, E. M. Cutting edge: control of CD8+ T cell activation by CD4+CD25+ immunoregulatory cells. J. Immunol. 167, 1137-1140 (2001).
7. Chai, J.-G. et al. CD4+CD25+ T cells as immunoregulatory T cells in vitro. Eur. J. Immunol. 32, 2365-2375 (2002).
8. Baecher-Allan, C., Brown, J. A., Freeman, G. J. & Hafler, D. A. CD4+CD25+ high regulatory cells in human peripheral blood. J. Immunol. 167, 1245-1253 (2001).
9. Jonuleit, H. et al. Identification and functional characterization of human CD4+CD25+ T cells with regulatory properties isolated from peripheral blood. J. Exp. Med. 193, 1285-1294 (2001).
10. Dieckmann, D., Plottner, H., Berchtold, S., Berger, T. & Schuler, G. Ex vivo isolation and characterization of CD4+CD25+ T cells with regulatory properties from human blood. J. Exp. Med. 193, 1303-1310 (2001).
11. Levings, M. K., Sangregorio, R. & Roncarolo, M.-G. Human CD25+CD4+ T regulatory cells suppress naive and memory T cell proliferation and can be expanded in vitro without loss of function. J. Exp. Med. 193, 1295-1302 (2001).
12. Stephens, L. A., Mottet, C., Mason. D: & Powrie, F. Human CD4+CD25+ thymocytes and peripheral T cells have immune suppressive activity in vitro. Eur. J. Immunol. 31, 1247-1254 (2001).
13. Taams, L. S. et al. Human anergic/suppressive CD4+CD25+ T cells: a highly differentiated and apoptosis-prone population. Eur. J. Immunol. 31, 1122-1131 (2001).
14. Van Maurik. A., Herber, M., Wood, K. J. & Jones, N. D. Cutting edge: CD4+CD25+ alloantigen-specific immunoregulatory cells that can prevent CD8+ T cell-mediated graft rejection: implications for anti-CD154 immunotherapy. J. Immunol. 169, 5401-5404 (2002).
15. Lin C.-Y., Graca, L., Cobbold, S. P. & Waldmann, H. Dominant transplantation tolerance impairs CD8+ T cell function but not expansion. Nat. Immunol. 3, 1208-1213 (2002).
16. Sakaguchi, S., Sakaguchi, N., Asano, M., Itoh, M. & Toda, M. Immunologic self-tolerance maintained by activated T cells expressing IL-2 receptor alpha-chains (CD25). Breakdown of a single mechanism of self-tolerance causes various autoimmune diseases. J. Immunol. 155, 1151-1164 (1995).
17. Taguchi, O. & Takahashi, T. Administration of anti-interleukin-2 receptor alpha antibody in vivo induces localized autoimmune disease. Eur. J. Immunol. 26, 1608-1612 (1996).
18. Suri-Payer. E., Amar, A. Z., Thornton, A. M. & Shevach, E. M. CD4+CD25+ T cells inhibit both the induction and effector function of autoreactive T cells and represent a unique lineage of immunoregulatory cells. J. Immunol. 160, 1212-1218 (1998).
19. Onizuka, S. et al. Tumor rejection by in vivo administration of anti-CD25 (interleukin-2 receptor alpha) monoclonal antibody. Cancer Res. 59, 3128-3133 (1999).
20. Golgher, D., Jones, E., Powrie, F., Elliott. T. & Gallimore, A. Depletion of CD25+ regulatory cells uncovers immune responses to shared murine tumor rejection antigens. Eur. J. Immunol. 32, 3267-3275 (2002).
21. Hall, B. M., Pearce, N. W., Gurley, K. E. & Dorsch, S. E. Specific unresponsiveness in rats with prolonged cardiac allograft survival after treatment with cyclosporine. III. Further characterization of the CD4+ suppressor cell and its mechanisms of action. J. Exp. Med. 171, 141-157 (1990).
22. Hara, M. et al. IL-10 is required for regulatory T cells to mediate tolerance to alloantigens in vivo. J. Immunol. 166, 3789-3796 (2001).
23. Sanchez-Fueyo, A., Weber, M., Domenig, C., Strom, T. B. & Zheng, X. X. Tracking the immunoregulatory mechanisms active during allograft tolerance. J. Immunol. 168, 2274-2281 (2002).
24. Gregori, S. et al. Regulatory T cells induced by 1-alpha,2,5-dihydroxyvitamin D3 and mycophenolate mofetil treatment mediate transplantation tolerance. J. Immunol. 167, 1945-1953 (2001).
25. Kingsley, C. I., Karim, M., Bushell, A. R. & Wood, K. J. CD25+CD4+ regulatory T cells prevent graft rejection: CTLA-4- and IL-10-dependent immunoregulation of alloresponses. J. Immunol. 168, 1080-1086 (2002).
26. Asseman, C., Mauze, S., Leach, M. W., Coffinan, R. L. & Powrie, F. An essential role for interleukin 10 in the function of regulatory T cells that inhibit intestinal inflammation. J. Exp. Med. 190, 995-1004 (1999).
27. Read, S., Malmstrom, V. & Powrie, F. Cytotoxic T lymphocyte-associated antigen 4 plays an essential role in the function of CD25+CD4+ regulatory cells that control intestinal inflammation. J. Exp. Med. 192, 295-302 (2000).
28. Liu, Z. et al. B7 interactions with CD28 and CTLA-4 control tolerance or induction of mucosal inflammation in chronic experimental colitis. J. Immunol. 167, 1830-1838 (2001).
29. Pearson, T. C., Madsen, J. C., Larsen, C. P., Morris. P. J. & Wood, K. J. Induction of transplantation tolerance in adults using donor antigen and anti-CD4 monoclonal antibody. Transplantation. 54, 475-483 (1992).
30. Taylor, P. A., C. J. Lees, and B. R. Blazar. The infusion of ex vivo activated and expanded CD4+CD25+ immune regulatory cells inhibits graft-versus host disease lethality. Blood 99:3493 (2002).
31. Taylor, P. A., R. J. Noelle, and B. R. Blazar. CD4+ CD25+ immune regulatory T cells are required for induction of tolerance to alloantigen via costimulatory blockade. J. Exp. Med. 193:1311 (2001).
32. Yamagiwa, S., G. D., S. Hashimoto, and D. A. Horwitz. A role for TGF-b in the generation and expansion of CD4+CD25+ regulatory T cells from human peripheral blood. J. Immunol. 166:7282 (2001).
33. Zheng, S. G., J. D. Gray, K. Ohtsuka, S. Yamagiwa, and D. A. Horwitz. Generation ex-vivo of TGF-b-producing regulatory T cells from CD4+ CD25− precursors. J. Immunol. Methods 169:4183 (2002).
34. Zheng, S. G., L. Meng, J. H. Wang, M. Watanabe, M. L. Barr, D. V. Cramer, J. D. Gray, and D. A. Horwitz. Transfer of regulatory cells generated ex vivo modifies graft rejection through induction of tolerogenic CD4+CD25+ cells in the recipient. Int. Immunol. 18:279 (2006).
35. Godfrey, W. R., D. J. Spoden, Y. G. Ge, S. R. Baker, B. Liu, B. L. Levine, C. H. June, B. R. Blazar, and S. B. Porter. Cord blood CD4+CD25+-derived T regulatory cell lines express FoxP3 protein and manifest potent suppressor function. Blood 105:750 (2005).
36. Bocek et al, J. Exp. Med., 199, 1619-1630 (2004).
37. Hong et al, PNAS, 102, 6449-6454 (2005).
38. New England J. Med., 352, 2598-2608 (2005)
39. Inaba, K., Inaba, M., Romani, N., Aya, H., Deguchi, M., Ikehara, S., Muramatsu, S., and Steinman, R. M. Generation of large numbers of dendritic cells from mouse bone marrow cultures supplemented with granulocyte/macrophage colony-stimulating factor. J. Exp. Med. 176, 1693-1702 (1992).
40. Yamaguchi, Y., Tsumura, H., Miwa, M., and Inaba, K. Contrasting effects of TGF-β and TNF-α on the development of dendritic cells from progenitors in mouse bone marrow. Stem Cells 15, 144-153 (1997).
41. Qin, S., Cobbold, S. P., Pope, H., Elliot, J., Kiossis, D., Davies, J., Waldmann, H. “Infectious” transplantation tolerance. Science, 259, 974-977 (1993).

1. An ex vivo method for generating a population of regulatory T cells (Treg) capable of suppressing rejection of an organ or tissue transplant from a donor animal in a recipient animal, which method comprises culturing CD4⁺ T cells from the recipient animal in the presence of IFN-γ plus either donor specific or third-party antigen presenting cells, and harvesting a population of Treg capable of suppressing rejection in the recipient animal.

2. A method according to claim 1 wherein the donor animal and the recipient animal are both human.

3. A method according to claim 1 wherein the antigen presenting cells are donor specific antigen presenting cells.

4. A method according to claim 3 wherein the donor specific antigen presenting cells are cells from the donor animal or cells pulsed with antigen from the donor animal.

5. A method according to claim 1 wherein total CD4⁺ T cells from the recipient animal are exposed in tissue culture to GM-CSF and TGF-β conditioned donor-type bone marrow dendritic cells (BM-DC) in the presence of IFD-γ for 5 to 10 days, and the cells are re-stimulated under identical conditions after a further 5 to 10 days.

6. A method of suppressing rejection of an organ or tissue transplant from a donor animal in a recipient animal comprising the following steps:
(i) obtaining a sample of CD4⁺ T-cells from the recipient animal;
(ii) culturing the said CD4⁺ T cells in the presence of IFN-γ plus either donor specific or third-party antigen presenting cells;
(iii) harvesting a population of Treg from said culture capable of suppressing rejection in the recipient animal; and
(iv) administering said Treg to the recipient animal.

7. A method according to claim 6 wherein the donor animal and recipient animal are both human.

8. A method according to claim 6 wherein the antigen cells are donor specific antigen presenting cells.

9. A method according to claim 8 wherein the donor specific antigen presenting cells are cells from the donor animal or cells pulsed with antigen from the donor animal.

10. A method according to claim 6 wherein total CD4⁺ T cells from the recipient animal are exposed in tissue culture to GM-CSF and TGF-β conditioned donor-type bone marrow dendritic cells (BM-DC) in the presence of IFD-γ for 5 to 10 days, and the cells are re-stimulated under identical conditions after a further 5 to 10 days.

11. A method according to claim 6 wherein the Treg are administered to the recipient animal intravenously.

12. A method according to claim 6 wherein the recipient is treated with additional immunosuppression or adjunctive therapy to attenuate any immediate rejection response.

13. A method according to claim 6 wherein the recipient is preconditioned to remove memory T cells prior to administration of the donor reactive Treg.

14. An ex vivo method for generating a population of Treg capable of suppressing an autoimmune condition in an animal wherein the animal mounts an immune reaction against an autoantigen, which method comprises culturing CD4⁺ T cells from the animal in the presence of cells presenting the autoantigen and IFN-γ and harvesting a population of autoantigen reactive Treg.

15. A method according to claim 14 wherein the animal is man.

16. A method according to claim 14 wherein the cells presenting the autoantigen are self antigen presenting cells plus autoantigen or antigen presenting cells isolated from a site of autoimmune attack.

17. A method of suppressing an autoimmune condition in an animal wherein the animal mounts an immune reaction against an autoantigen comprising the following steps:
(i) obtaining a sample of CD4⁺ T-cells from the animal;
(ii) culturing the said CD4⁺ T cells in the presence of cells presenting the autoantigen and IFN-γ;
(iii) harvesting a population of autoantigen reactive Treg from said culture; and
(iv) administering said Treg to the animal.

18. A method according to claim 17 wherein the animal is man.

19. A method according to claim 17 wherein the cells presenting the autoantigen are self antigen presenting cells plus autoantigen or antigen presenting cells isolated from a site of autoimmune attack.

20. A method according to claim 17 wherein the Treg are administered to the recipient animal intravenously.

21. A method according to claim 17 wherein the autoimmune condition is rheumatoid arthritis, multiple sclerosis, insulin dependent diabetes melitus or inflammatory bowel disease.

22. A method according to claim 17 wherein the animal is subjected to pre-conditioning to deplete autoantigen reactive T cells prior to administration of the autoantigen reactive Treg.
